(19) United States
(12) Patent Application Publication
(10) Pub. No.: US 2016/0015543 A1
(43) Pub. Date: Jan. 21, 2016

(54) TREATMENT DEVICE FOR A BLOOD CIRCULATION CONDUIT

(71) Applicant: CORMOVE, Bornel (FR)

(72) Inventors: Eric Perouse, Paris (FR); Witold Styrc, Kopstal (LU)

(21) Appl. No.: 14/799,734

(22) Filed: Jul. 15, 2015

(30) Foreign Application Priority Data
Jul. 17, 2014 (FR) FR1456889

(51) Int. Cl. A61F 2/962; A61F 2/91; A61F 2/958; A61F 2/844

(52) U.S. Cl. CPC A61F 2/962; A61F 2/844; A61F 2/91; A61F 2/958; A61F 2002/9511

(57) ABSTRACT

The device includes:

a tubular endoprosthesis with a central axis, and a release system including a capsule defining an inner volume receiving the tubular endoprosthesis in its contracted condition. The capsule is displaceable with respect to the tubular endoprosthesis along the central axis between a position for covering the tubular endoprosthesis and a position for releasing the tubular endoprosthesis.

The device includes a mechanism for opening the capsule able to be maneuvered in the covering position in order to have the capsule pass from a closed configuration for retaining the tubular endoprosthesis to an open configuration radially expansed for releasing the tubular endoprosthesis. The tubular endoprosthesis is able to be kept at least partly retracted in the inner volume upon passing of the capsule from its closed configuration to its open configuration.

This claims the benefit of French Patent Application 14 56889, filed Jul. 17, 2014 and hereby incorporated by reference herein.

The present invention relates to a device for treating a blood flow conduit, including:

a tubular endoprosthesis with a central axis, which may be deployed between a contracted condition and a deployed condition,

a system for releasing the tubular endoprosthesis including a capsule defining an inner volume receiving the tubular endoprosthesis in its contracted condition, the capsule being displaceable relatively to the tubular endoprosthesis along the central axis between a position for covering the tubular endoprosthesis and a position for releasing the tubular endoprosthesis.

### BACKGROUND

This device is notably applied to the treatment of heart valves.

The heart includes valves which are present at the outlet of the right ventricle (pulmonary and tricuspid valve) and of the left ventricle (aortic and mitral valve).

These valves ensure a one-way circulation of the blood flow, avoiding blood backflow at the end of the ventricular contraction.

However, diseases and malformations affect the good operation of the valves.

In particular, the latter may suffer from calcification thus allowing backflow or regurgitation towards the ventricle or the atrium having expelled the blood flow. The problem of regurgitation leads to abnormal expansion of the ventricle which finally produces a heart failure. In certain cases, the valve comprises a number of sheets below that which is generally observed for this type of valve, which may be detrimental to its operation in the long term.

In order to treat this type of disease surgically, it is known to implant an endovalve between the sheets of the affected native valve. This endovalve comprises a tubular endoprosthesis formed by a self-extensible trellis and a flexible obturator or valve most often made in a tissue of animal origin. The flexible obturator is permanently fixed in the endoprosthesis.

Such endovalves are implantable via an endoluminal route, which considerably limits the risks associated with the implantation of the valve, notably in terms of mortality.

In order to promote implantation via the endoluminal route, it is necessary to reduce as much as possible the congestion of the endoprosthesis bearing the valve during its path through the blood flow conduits as far as the valve to be treated.

For this purpose, the endoprosthesis in its contracted configuration is accommodated in a capsule located in the vicinity of the nose of the release system. US 2005/090834 describes a system for releasing an endoprosthesis of the aforementioned type.

In order to deploy the endoprosthesis, the capsule is axially moved relatively to the endoprosthesis. When the latter is self-expansible, it deploys radially in order to come into contact with the walls of the conduit in which it is implanted.

Such a release system does not give entire satisfaction. Indeed, the axial withdrawal of the capsule relatively to the endoprosthesis is sometimes difficult to achieve, notably when the capsule firmly clasps the endoprosthesis. In this case, the radial forces applied by the capsule on the endoprosthesis increase axial friction between the capsule and the endoprosthesis.

In order to limit this friction, it is possible to slightly increase the radial extension of the capsule. However, this increases the dimensions of the release system, and complicates its introduction into the blood network of the patient.

### SUMMARY OF THE INVENTION

It is an object of the present invention to provide a particularly compact treatment device, but which remains nevertheless very simple to set into place in a blood flow conduit.

For this purpose, the object of the invention is a device of the aforementioned type, characterized in that the device includes a mechanism for opening the capsule able to be maneuvered in the covering position in order to have the capsule pass from a closed configuration for retaining the tubular endoprosthesis to an open configuration radially expansed for releasing the tubular endoprosthesis, the tubular endoprosthesis being able to be kept at least partly retracted in the inner volume during the passage of the capsule from its closed configuration to its open configuration.

The device according to the invention may comprise one or several of the following features, taken individually or according to any technically possible combination:

the opening mechanism is able to release at least one side passage through the capsule during the passing of the capsule from its closed configuration to its open configuration.

the edges of the side passage in the open configuration are located in contact with each other in the closed configuration.

the opening mechanism includes at least one member for cutting out the capsule, the side passage being generated by cutting out the capsule during the maneuver of the opening mechanism.

the capsule delimits the side passage, the opening mechanism including at least one member for closing the releasable side passage during the maneuvering the opening mechanism.

the tubular endoprosthesis is able to remain confined in the inner volume out of the side passage during the maneuvering of the opening mechanism.

the tubular endoprosthesis may be spontaneously deployed from its contracted condition to its deployed condition, the release system including an assembly for retaining the tubular endoprosthesis, the retaining assembly being able to maintain at least one portion of the tubular endoprosthesis in its contracted condition, upon passing of the capsule from its closed configuration to its open configuration.

the retaining assembly includes at least one releasable wire-shaped bond defining a retaining loop surrounding the tubular endoprosthesis.

the retaining assembly includes a hollow support delimiting a retaining aperture through which the wire-shaped bond is engaged, the wire-shaped bond having a control segment which may be actuated by a user from a proximal end of the support.

the tubular endoprosthesis is spontaneously maintained in its contracted configuration, the release system including a balloon for radially deploying the tubular endoprosthesis towards its deployed configuration.

the capsule in the closed configuration clasps the tubular endoprosthesis in the contracted condition, the axial force for retaining the tubular endoprosthesis in the capsule in the closed configuration being greater than the axial force for retaining the tubular endoprosthesis in the capsule in the open configuration.

Another object of the present invention is a method for releasing an endoprosthesis comprising the following steps:

providing a device as described above;

opening the capsule by means of the opening mechanism for having the capsule pass from a closed configuration for retaining the endoprosthesis to an open configuration radially expansed for releasing the endoprosthesis,

the endoprosthesis being kept in the inner volume by the retaining assembly upon passing of the capsule from its closed configuration to its open configuration.

### BRIEF DESCRIPTION OF THE DRAWINGS

The invention will be better understood upon reading the description which follows, only given as an example, and made with reference to the appended drawings, wherein:

FIG. 1 is a side view of a first treatment device according to the invention, the capsule occupying a closed configuration receiving the endoprosthesis;

FIG. 2 is a partial sectional view along a middle axial plane of the first treatment device;

FIG. 3 is an end view from the distal end of the first device of FIG. 1, the nose having been withdrawn;

FIG. 4 is a view similar to FIG. 1, after opening the capsule, and before its axial displacement with respect to the endoprosthesis;

FIG. 5 is an end view of FIG. 4;

FIG. 6 is a view similar to FIG. 4, after axial displacement of the capsule relatively to the endoprosthesis;

FIG. 7 is a view similar to FIG. 6, after withdrawal of the assembly for retaining the endoprosthesis and the capsule;

FIG. 8 is a view similar to FIG. 1 of a second treatment device according to the invention;

FIG. 9 is a view similar to FIG. 2 of a third treatment device according to the invention.

### DETAILED DESCRIPTION

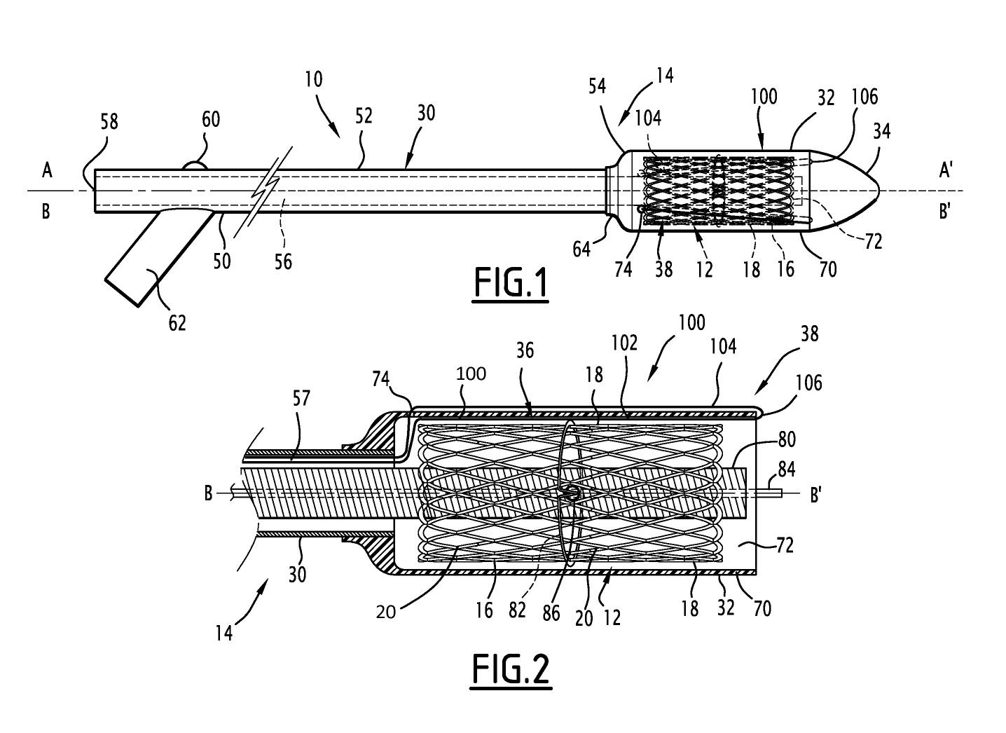

A first treatment device 10 according to the invention is illustrated in FIGS. 1 to 7. This device 10 is intended to be introduced into a blood flow conduit of a patient.

The device 10 includes a prosthetic implant 12, intended to be placed in the blood flow conduit, and a system 14 for releasing the prosthetic implant 12.

In this example, the prosthetic implant 12 is an endovalve. The implant 12 includes a tubular endoprosthesis 16, and a prosthetic valve 18 mounted in the tubular endoprosthesis 16.

As is visible in FIG. 7, the tubular endoprosthesis 16 is formed by a tubular open-worked frame 20 with the central axis A-A′ comprising a trellis of wires which have spring properties. The frame 20 is for example obtained by braiding at least one wire of stainless steel, from a shape memory alloy, or from a polymer.

Alternatively, the frame 20 is obtained by cutting out with a laser a tube or another technique.

The frame 20 defines an inner peripheral surface and an outer peripheral surface around the axis A-A′.

The inner peripheral surface defines a central passage 22 for blood flow. The outer peripheral surface is intended to be applied at least partly against a wall of the blood flow conduit and/or against the native sheets.

The frame 20 of the endoprosthesis 12 may be deployed between a contracted condition, in which it has a smaller diameter, with view to its introduction into the conduit, and an expansed condition, making up its rest condition, in which it has a large diameter.

In the example illustrated in FIGS. 1 to 4, the frame 20 may be spontaneously deployed between its contracted condition and its expansed condition.

The valve 18 is for example made on the basis of a native valve of an animal like a pig or other animals. Alternatively, it is made on the basis of natural tissues such as bovine or sheep pericardium or on the basis of synthetic tissues.

Conventionally, the valve 18 comprises a tubular base attached on the inner surface of the frame 20, and flexible sheets for obturating the central passage 22, for example three in number. The sheets may be displaced radially towards the axis of the passage 22 between an obturation position, in which they substantially prevent totally the passing of the blood through the passage 22, and a position for clearing the passage 22 in which they are substantially flattened against the inner surface of the endoprosthesis 16 and in which they let through the blood through the passage 22.

With reference to FIG. 1, the release system 14 includes an external sheath 30 with a central axis B-B′, a capsule 32 for receiving the implant 12, positioned at the distal end of the external sheath 30, and a nose 34 for distal obturation of the capsule 32.

With reference to FIG. 2, the release system 14 further includes a releasable assembly 36 for retaining the implant 12 in the release system 14, and according to the invention, a mechanism 38 for longitudinally opening the capsule 32 in order to have it pass from a closed configuration to an open configuration.

As visible in FIG. 1, the external sheath 30 includes a proximal segment 50 intended to be maintained outside the patient, an intermediate segment 52 and a head 54, the segment 52 and the head 54 being intended to be introduced into the blood network of the patient. The sheath 30 defines a central passage 56 for flow of the releasable retention assembly 36 and a peripheral flow passage 57 of the opening mechanism 38 which is distinct or coincides with the central passage 56.

The proximal segment 50 delimits in this example a proximal axial aperture 58 through which opens the central passage 56. It includes a member 60 for maneuvering the releasable retention assembly 36 and a branch 62 for maneuvering the opening mechanism 38.

The intermediate segment 52 is elastically deformable by flexure in order to circulate in the blood network of the patient. For example it has a maximum diameter comprised between 3 mm and 7 mm.

The head 54 is positioned at the distal end of the external sheath 30. The central passage 56 axially opens through the head 54.

In this example, the head 54 has a radial extension greater than that of the intermediate segment 52 and defines a shoulder 64 with the intermediate segment 52.

The capsule 32 is added onto the external sheath 30 at its distal end. Alternatively, the capsule 32 is made in the same material with the external sheath 30.

The capsule 32 includes a tubular sleeve 70 with a central axis B-B′ defining an inner volume 72 for receiving the implant 12.

In this example, the tubular sleeve 70 is made in plastic material, for example in polytetrafluoroethylene.

It has a wall thickness of less than the wall thickness of the external sheath 30. This wall thickness is for example less than 0.15 mm, and notably comprised between 0.5 mm and 0.1 mm, or between 0.1 mm and 0.05 mm.

The external peripheral surface of the capsule 32 is flush with the external peripheral surface of the external sheath 30 at the head 54.

The length of the capsule 32, taken along the axis B-B′ is greater than that of the implant 12. Thus, the implant 12 is able to be totally contained in the capsule 32.

In this example, the wall of the capsule 32 is solid, except for through-holes 74 for letting through the opening mechanism 38, which open into the inner volume 72.

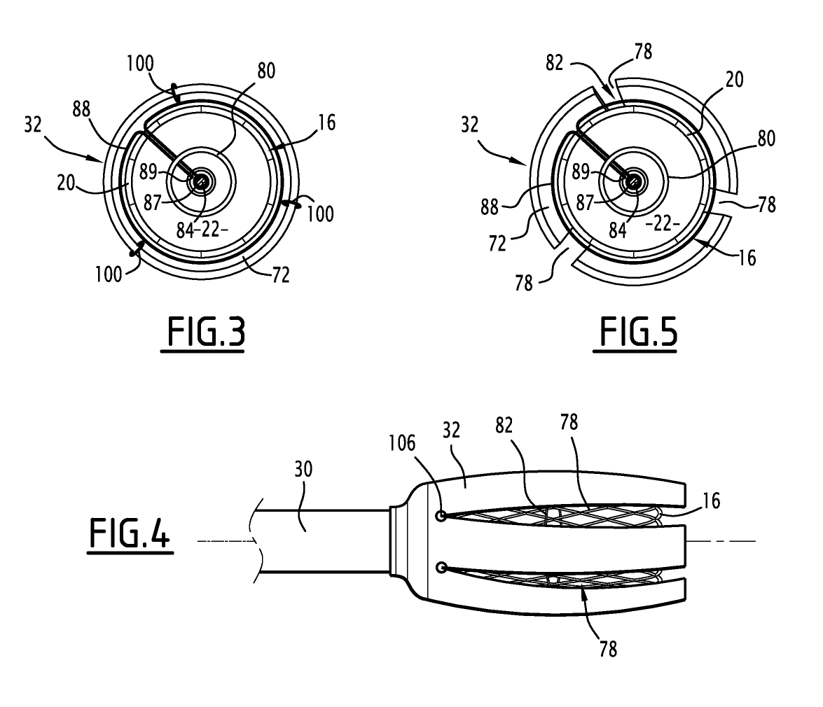

The capsule 32 is able to pass from a closed configuration for retaining the implant 12, visible in FIGS. 2 to 3, to an open configuration radially expansed for releasing the implant 12, visible in FIGS. 4 and 5.

In the closed configuration, the capsule 32 has a minimum transverse extension. Its maximum outer diameter is for example less than 9 mm and is notably comprised between 9 mm and 3 mm.

The endoprosthesis 16 is maintained in its contracted condition, with its outer surface in contact with the inner surface of the capsule 32. The capsule 32 clasps the endoprosthesis 16.

In the open configuration, side passages 78 have been opened in the capsule 32 by the opening mechanism 38. The side passages 78 extend along the capsule 32, advantageously parallel to the axis A-A′.

The transverse extension of the capsule 32 is then increased. Its maximum outer diameter is for example greater than more than 150% of the outer diameter in the closed configuration.

According to the invention, the endoprosthesis 16 is maintained at least partly in its contracted condition via the releasable retention assembly 36. The inner surface of the capsule 32 is at least partly moved away from the outer surface of the tubular endoprosthesis 16.

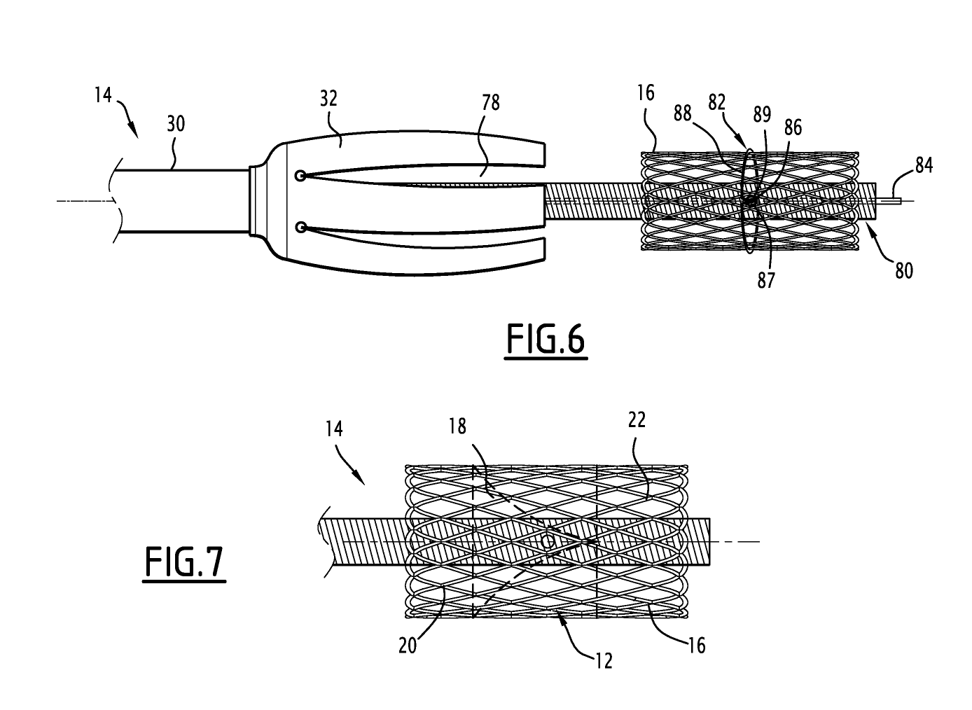

The external sheath 30 and the external sheath 32 are movable together along the axis A-A′ with respect to the implant 12, between a distal position for covering the implant 12, visible in FIG. 2, in which the implant 12 is received into the inner volume 72 and a proximal position for releasing the implant 12, visible in FIG. 6, in which the capsule 32 is axially shifted with respect to the implant 12.

The releasable attention assembly 36 is for example as described in French application FR 2 863 160. It includes in this example a hollow support 80 positioned through the inner volume 72, a wire-shaped retention bond 82, partly surrounding the endoprosthesis 16, and a rod 84 for retaining the wire-shaped bond.

The support 80 comprises a hollow flexible metal tube. The internal diameter of the tube is adapted for slipping the latter onto a wire-shaped surgical guide (not shown) installed in the patient, before setting into place the endoprosthesis 16 in a blood vessel of this patient.

The support 80 extends longitudinally between a distal end intended to be implanted into the blood vessel and a proximal end intended to be accessible for a surgeon.

A distal retention aperture 86 associated with the wire-shaped bond 82 is made laterally in the support 80.

The retention rod 84 is positioned in the support 80.

The rod 84 is translationally movable in the support 80, between a retention position, in which it is placed facing the retention aperture 86, and a release position, in which it is placed away from the retention apertures 86.

In the example illustrated in FIG. 3, the assembly 36 comprises a single wire-shaped bond 82. Alternatively, the assembly 36 comprises several wire-shaped bonds 82.

As visible in FIGS. 5 and 6, the wire-shaped bond 82 comprises here a single strand, which includes an end loop 87, a tightening loop 88, and a control segment 89.

The end loop 87 is positioned at a distal end of the strand. It is formed with a closed loop. The rod 84 is engaged into the loop 87, when the rod 84 is in its retaining position.

The loop 87 is moreover deformable so that its width, when it is deformed is substantially equal to twice the width of the strand.

The loop 87 is connected to the tightening loop 88 through a segment engaged into the distal retention aperture 86.

The tightening loop 88 is formed with a segment of the strand, engaged around the endoprosthesis 16.

The tightening loop 88 extends between a retention end connected to the loop 87 and a tightening end connected to the control segment 89 and engaged into the retention aperture 86. This tightening loop 88 attaches the endoprosthesis 16 to the support 80 in the vicinity of the distal end of this support 80.

The active length of the tightening loop 88 is variable, so that it controls the deployment of the endoprosthesis 16 with respect to the support 80, as will be described later on.

The control segment 89 extends from the support 80 between the distal retention aperture 86 and the maneuvering member 60 onto which it is engaged.

The length of the control segment 89 engaged onto the maneuvering member 60 is variable and controls the length of the tightening loop 88.

Thus, an increase in the length of the control segment engaged onto the maneuvering member 60 causes a corresponding reduction in the active length of the tightening loop 88, and subsequently, tightening of the endoprosthesis 16 against the support 80, at the tightening loop 88.

When the endoprosthesis 16 is in its contracted condition against the support 80, the control segment 89 is in a tensioned position.

Conversely, a decrease in the length of the control segment engaged onto the maneuvering member 60 causes an increase in the active length of the tightening loop 88 and subsequently, the deployment of the endoprosthesis 16 away from the support 80, at the tightening loop 88.

When the endoprosthesis 16 is in its expansed condition, the control segment 89 is in a relaxed position.

In this example, with reference to FIGS. 1 and 2, the mechanism 38 for opening the capsule 32 includes at least one member 100 for longitudinally cutting out the capsule, able to be controlled from the proximal end of the release system 14 in order to open a side passage 78 through the capsule 32 (visible in FIG. 4).

The opening mechanism 38 includes here several parallel cutting-out members 100, each able to open a longitudinal passage 74 through the wall of the capsule 32.

The longitudinal cutting-out member 100 is for example formed with a cutting-out wire folded as a loop around the wall of the capsule. The cutting-out wire includes an inner strand 102 and an outer strand 104 connected together to their distal end 106.

The inner strand 102 includes a distal segment, substantially parallel to the axis A-A′, positioned in the inner volume 72, between the endoprosthesis 16 and the capsule 32, and a proximal segment engaged into the sheet 30 as far as the branch 62.

The outer strand 104 includes a distal segment positioned outside the capsule 32 substantially parallel to the axis B-B′, along its outer peripheral surface between the distal edge of the capsule 32 and a through-aperture 74. It includes an intermediate segment engaged into the inner volume 72 through the aperture 74, and a proximal segment engaged into the sheath 30 as far as the branching 62.

The longitudinal cutting-out member 100 is able to be drawn in a proximal way from the branching 62 outside the body of the patient between a distal position, visible in FIG. 1, in which the distal end 106 is initially positioned around the distal edge of the capsule 32, and a proximal position, visible in FIG. 4, in which the distal end 106 is moved towards a proximal edge of the capsule 32 and has longitudinally cut out the capsule 32 in order to generate a side passage 78 opening into the inner volume 72.

The operation of the first treatment device 10 will now be described.

Initially, the release system 14 is provided, with its releasable retention assembly 36. The support 80 protrudes in a proximal way beyond the proximal edge of the capsule 32. The tightening loop 88 protrudes out of the retention aperture 86. The capsule 32 is in its closed configuration.

The operator selects a suitable implant 12 in order to be implanted in the patient.

He/she places the implant 12 in its expansed condition around the support by introducing the support into the central passage 22. Next, he/she places the tightening loop 88 around the tubular endoprosthesis 16 so that it surrounds the endoprosthesis 16.

It then actuates the maneuvering member 60 for reducing the active length of the tightening loop 88 and for letting through the endoprosthesis 16 at least partly in its contracted condition.

Next, he/she introduces the endoprosthesis 16 into the inner volume 72 of the capsule 32 by compressing its proximal edge and by relatively displacing the capsule 32 and the support 80. The capsule 32 then occupies its distal position for covering the endoprosthesis 16, as visible in FIG. 1.

The endoprosthesis 16 is then maintained in a highly confined way in the inner volume 72 of the capsule 32 occupying its closed configuration. The radial dimensions of the treatment device 10 are then a minimum.

Next, the operator inserts a surgical guide into the body of the patient via an endoluminal route, as far as the implantation site of the implant 12. He/she engages the treatment device 10 around the surgical guide in order to introduce it into the body of the patient and brings the capsule 32 as far as the implantation site by maintaining the maneuvering member 60, the branching 62 and the proximal end of the external sheath 30 outside the patient.

This having been done, the operator actuates the opening mechanism 38 in order to have the capsule 32 pass into its open configuration.

In the configuration illustrated by FIGS. 1 to 7, the operator pulls on the cutting-out member 100 through the branching 62. He/she displaces the cutting-out member 100 from its distal position to its proximal position in order to bring the distal end 106 closer to the proximal edge of the capsule 32.

This displacement longitudinally cuts out the capsule 32 and generates at least one side passage 78.

The capsule 32 expands, so that the inner volume 72 increases radially. According to the invention, the retention assembly 36 maintains the endoprosthesis 16 at least partly in its contracted condition.

The endoprosthesis 16 therefore remains contained in the inner volume 72, without protruding out of the side passages 78 open in the capsule 32.

The operator may then displace the capsule 32 with respect to the endoprosthesis 16 in order to uncover the endoprosthesis 16. At least one passage 78 having been opened laterally, the capsule 32 in the open configuration no longer clasps the endoprosthesis 16 like in the closed configuration.

The axial displacement of the capsule 32 with respect to the endoprosthesis 16 is therefore easy to achieve and does not require overcoming a significant frictional force.

However, by the presence of the active releasable retention assembly 36 during the opening of the capsule 32, the endoprosthesis 16 remains at least partly in its contracted condition. It may therefore be positioned accurately and reversibly at its implantation site.

Subsequently, the operator actuates the maneuvering member 60 in order to release the wire-shaped bonds and to increase the active length of the tightening loop 88. The endoprosthesis 16 deploys radially and comes into contact with the wall delimiting the blood flow conduit and/or with the sheets of the native valve.

If the operator is not satisfied by the positioning of the endoprosthesis 16, he/she again actuates the maneuvering member 60 for reducing the active length of the tightening loop 88 and retracting at least partly the endoprosthesis 16.

When the operator estimates that the endoprosthesis 16 is properly positioned, he/she releases the end loop 87 of the tightening loop 88 by withdrawing the retaining rod 84, as illustrated in FIG. 7. Next, he/she extracts the retaining rod 84, the wire-shaped bond 82 and the support 80 out of the patient.

The device 10 according to the invention is therefore particularly compact, while allowing simple release of the implant 12 by the opening of the capsule 32, the deployment of the implant 12 remaining under the control of the releasable retention assembly 36.

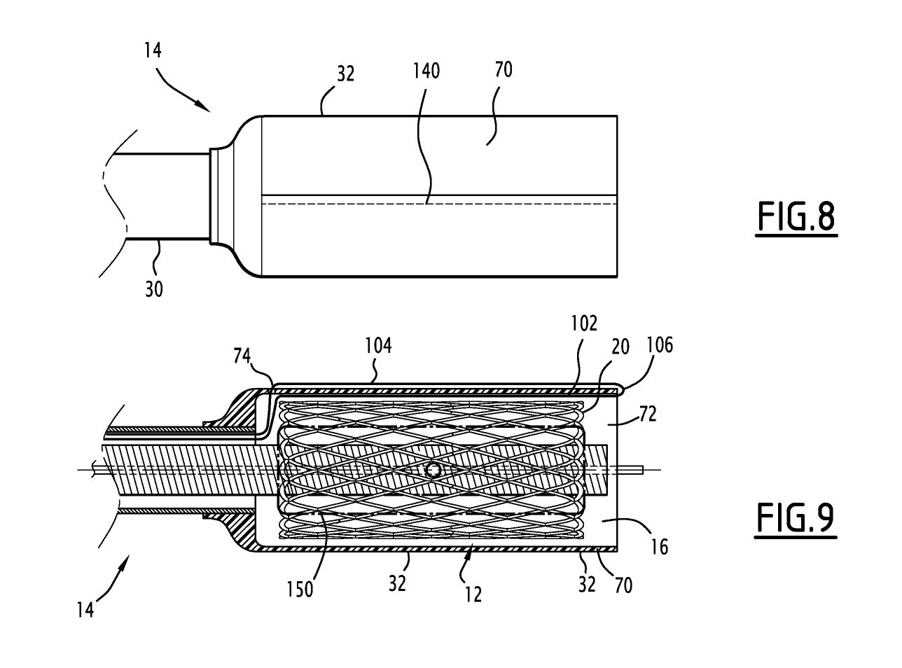

In an alternative illustrated in FIG. 8, the capsule 32 is formed with an existing side passage 78. The edges of the side passage 78 are assembled onto each other by the opening mechanism 38.

For this purpose, a wire-shaped member 140 is for example sewn between the edges of the side passage 78. The wire-shaped member 140 is withdrawn so as to have the capsule 32 pass from the closed configuration to the open configuration by opening the side passage 78.

In another alternative, illustrated in FIG. 9, the endoprosthesis 16 is maintained at rest in its contracted condition. A balloon 150 is inserted between the support 80 and the internal surface of the endoprosthesis 16.

As previously, the operator longitudinally opens the capsule 32 by means of the opening mechanism 38, and then uncovers the endoprosthesis 16 by displacing the external sheath 30 with respect to the endoprosthesis 16.

As the endoprosthesis 16 cannot be spontaneously deployed, it remains in its contracted condition.

The operator then inflates the balloon 150 at the selected implantation site in order to have the endoprosthesis 16 pass into its deployed condition.

The endoprosthesis 16 is maintained in an extremely compact way in the capsule 32 during its transport towards the implantation site in the release system 14, while being easily extractible out of the capsule 32 once the latter is opened. As the endoprosthesis 16 cannot be spontaneously deployed, its deployment at the implantation site is controlled and is posterior to the opening of the capsule 32.

1. A device for treating a blood circulation conduit including:
a tubular endoprosthesis with a central axis, deployable between a contracted condition and a deployed condition,
a system for releasing the tubular endoprosthesis including a capsule defining an inner volume receiving the tubular endoprosthesis in its contracted condition, the capsule being displaceable with respect to the tubular endoprosthesis along the central axis between a position for covering the tubular endoprosthesis and a position for releasing the tubular endoprosthesis;
a mechanism for opening the capsule able to be maneuvered in the covering position for having the capsule pass from a closed configuration for retaining the tubular endoprosthesis to an open configuration radially expansed for releasing the tubular endoprosthesis, the tubular endoprosthesis being able to be kept at least partly retracted in the inner volume during the passing of the capsule from its closed configuration to its open configuration.

2. The device according to claim 1, wherein the opening mechanism is able to release at least one side passage through the capsule upon passing of the capsule from its closed configuration to its open configuration.

3. The device according to claim 2, wherein the edges of the side passage in the open configuration are located in contact with each other in the closed configuration.

4. The device according to claim 2, wherein the opening mechanism includes at least one member for cutting out the capsule, the side passage being generated by cutting out the capsule during the maneuvering of the opening mechanism.

5. The device according to claim 2, wherein the capsule delimits the side passage, the opening mechanism including at least one member for closing the side passage, releasable during the maneuvering of the opening mechanism.

6. The device according to claim 2, wherein the tubular endoprosthesis remains confined in the inner volume out of the side passage during the maneuvering of the opening mechanism.

7. The device according to claim 1, wherein the tubular endoprosthesis is able to be spontaneously deployed from its contracted condition to its deployed condition, the release system including an assembly for retaining the tubular endoprosthesis, the retention assembly being able to maintain at least one portion of the tubular endoprosthesis in its contracted condition, upon passing of the capsule from its closed configuration to its open configuration.

8. The device according to claim 7, wherein the retention assembly includes at least one releasable wire-shaped bond defining a retention loop surrounding the tubular endoprosthesis.

9. The device according to claim 8, wherein the retention assembly includes a hollow support delimiting a retention aperture through which the wire-shaped bond is engaged, the wire-shaped bond having a control segment which may be actuated by a user from a proximal end of the support.

10. The device according to claim 1, wherein the tubular endoprosthesis is spontaneously maintained in its contracted configuration, the release system including a balloon for radial deployment of the tubular endoprosthesis towards its deployed configuration.

11. The device according to claim 1, wherein the capsule in the closed configuration clasps the tubular endoprosthesis in the contracted condition, the axial force for retaining the tubular endoprosthesis in the capsule in the closed configuration being greater than the axial force for retaining the tubular endoprosthesis in the capsule in the open configuration.

12. Method for releasing an endoprosthesis comprising the following steps:
providing a device according to claim 1;
opening the capsule by means of the opening mechanism for having the capsule pass from a closed configuration for retaining the endoprosthesis to an open configuration radially expansed for releasing the endoprosthesis,
the endoprosthesis being kept in the inner volume by the retaining assembly upon passing of the capsule from its closed configuration to its open configuration.
